(19) United States
(12) Patent Application Publication
(10) Pub. No.: US 2019/0259489 A1
(43) Pub. Date: Aug. 22, 2019

(54) METHOD AND SYSTEM FOR SUPPORTING A USER IN THE SELECTION OF FOOD

(71) Applicant: TELECOM ITALIA S.p.A., Milano (IT)

(72) Inventors: Gianluca DE PETRIS, Pisa (IT); Roberta Rita MUOLO, Pisa (IT); Claudio PETRAZZUOLO, Pisa (IT); Giovanna SACCHI, Pisa (IT)

(73) Assignee: TELECOM ITALIA S.p.A., Milano (IT)

(21) Appl. No.: 16/333,459

(22) PCT Filed: Sep. 21, 2016
(86) PCT No.: PCT/EP2016/072390

§ 371 Date: Mar. 14, 2019

(51) Int. Cl. G16H 20/60; G09B 19/00; G06F 17/16

(52) U.S. Cl. CPC G16H 20/60; G06F 17/16; G09B 19/0092

(57) ABSTRACT

A system for supporting a user in the selection of food includes a processing module that determines a diet pattern of the user. The diet pattern includes nutritional information related to a number of macronutrients or micronutrients or bioactive compounds into which food may be decomposed and corresponding values of daily intakes. The diet pattern includes a number of food rules, each food rule including at least one food category and, for each food category, a respective frequency of intake and a respective reference quantity of intake. The system receives from the user information indicating a target food, and computes a compliance score of the target food with the diet pattern and notifying the user of the compliance score.

### TECHNICAL FIELD

The present invention relates to the field of the analysis and control of dietary habits. In particular, the present invention relates to a system and method for supporting a user in the selection of food.

### BACKGROUND ART

Systems and methods are known that allow monitoring a user's adherence to a specific diet in order, for instance, to treat or prevent particular medical conditions, such as obesity or cardiovascular diseases. These systems usually exploit data supplied by the user through personal devices, such as computer devices and/or user's wearable devices, and sensors capable of tracking user's health parameters (weight, blood pressure, hydration, heart rate, etc.).

U.S. Pat. No. 7,297,109 discloses a method, system and computer program product for remotely measuring one's adherence to a diet program while making it convenient and easy to place food orders, and learn about and try new foods that are acceptable to the diet program. The system includes a user system with a processor, memory and at least one user interface device, a server system with a processor and memory, and a food delivery system coupled to the server system over the network. The system of U.S. Pat. No. 7,297,109 provides for generating a diet program (meal or diet plan) for a user and a program adherence value that indicates how well the patient is adhering to the program. In particular, the system determines food intake and prepares a food list for the user according to the program and on the basis of specific goal achievements (e.g. reducing fat intake to 20%, losing 10 pounds). Eventually, the food delivery system may prepare a food order according to the food list.

US 2015/0093725 A1 discloses a system and method for providing dietary guidance. The method includes receiving a selection of a health program for an individual, the health program including a dietary regimen, measuring the individual's caloric expenditure and change in body composition or body mass during the individual's participation in the health program, determining adherence to the health program based on the measured caloric expenditure or the measured change in body composition or body mass, identifying a modification to the health program, and informing the individual of the modification. The modification can include nutritional supplements, meals or recipes having a nutritional and/or caloric content tailored to assist the individual in meeting his or her health goals. The method can further include predicting an expected change in body composition or body mass based on the health program and based on the individual's gender, age, height, weight, and other factors.

### SUMMARY OF THE INVENTION

The Applicant has noticed that according to the systems described above, the user is associated with a diet program that she/he shall follow. Therefore, the user is subject to constraints and prohibitions regarding her/his meals. This situation is disadvantageous for many users who may not be able to eat the foods prescribed by the diet. For instance, these users can not have their meals at home during the day or may not be able to find the prescribed foods in a restaurant's menu. Moreover, a user may get bored with food prohibitions.

In view of the above, the Applicant has tackled the problem of providing a system and method for supporting a user in the selection of food which allows overcoming the drawbacks underlined above. In particular, the Applicant has tackled the problem of providing a system and a method for supporting a user in the selection of food which is more flexible than the systems and methods described above, i.e. which leaves a certain degree of freedom to the user in selecting the food while, at the same time, helping the user to adopt healthy dietary habits compliant to a personalized indication possibly given by an experienced nutritionist, or chosen by the user itself according to his/her needs (e.g. user's conditions such as a cardiovascular disease), his/her objectives (e.g. losing weight), and imposed restrictions (e.g. allergies and intolerances).

According to embodiments of the present invention, the problem above is solved by a system and a method for supporting a user in the selection of food that receives from the user information about a given food (e.g. a food that the user is eating or purchasing or is going to eat or purchase) and analyses this information on the basis of a reference dietary model (which will be referred to in the following description and in the claims as “diet pattern”) associated with the user and tailored on the basis of his/her nutritional needs. A score indicating the compliance of the food to the reference diet pattern is also calculated.

In the following description and in the claims, the term “macronutrient” will refer to a class of basic nutrients that provide energy to an organism or, in other words, that are required in high quantities by an organism for maintenance and accretion; examples of macronutrients for an animal (in particular, human) organism are carbohydrates, proteins and fats.

The term “micronutrient” will refer to nutrients required by an organism in small quantities so as to enable a range of physiological functions; examples of micronutrients are vitamins and minerals.

At last the term “bioactive compound” is a compound that can have influence on health, such as omega-3 and omega-6 for preventing cardiovascular diseases, and beta-glucan for preventing vascular diseases.

According to a first aspect, the present invention provides a system for supporting a user in the selection of food, the system comprising a processing module configured to:
determine a diet pattern of the user, the diet pattern comprising nutritional information related to a number of macronutrients or micronutrients or bioactive compounds into which food may be decomposed and corresponding values of daily intakes, the diet pattern further comprising a number of food rules, each food rule comprising at least one food category and, for each food category, a respective frequency of intake and a respective reference quantity of intake; and
receive from the user information indicating a target food, compute a compliance score of the target food with the diet pattern and notifying the user of the compliance score.

Preferably, the system further comprises a diet pattern database comprising a set of pre-determined diet patterns, wherein the processing module is further configured to
receive a user's profile comprising user-related information, the user-related information comprising one or more of: gender, height, weight, body mass index, age, diseases, intolerances, allergies;
select one diet pattern of the set of pre-determined diet patterns; and
customize the selected diet pattern on the basis of the user's profile.

Preferably, the system further comprises a food database comprising a number of entries relating to foods and, for each entry, the respective food category and nutritional information comprising quantities of the number of macronutrients or micronutrients or bioactive compounds for a reference amount of the respective food.

Preferably, the system further comprises a user database configured to store:
a food diary of the user comprising information about the foods consumed by the user subdivided in a number of food categories; and
a user nutrition track table comprising nutritional information of each food consumed by the user, the nutritional information comprising the macronutrients or micronutrients or bioactive compounds.

Preferably, the processing module is further configured to, upon reception from the user of the information indicating a target food, wherein the information indicating a target food comprises a target food intake:
access the food database to get the food category of the target food and the nutritional information related to the reference amount of the target food;
compute the nutritional information related to the target food intake; and
access the user database to update the food diary and the user nutrition track table with the information indicating the target food and the computed nutritional information.

Preferably, the processing module is further configured to compute the compliance score as product of a partial compliance score indicating the compliance of the target food with respect to the nutritional information of the user diet pattern and a weight value indicating whether the target food fulfils the food rules of the user diet pattern.

Preferably, the processing module is further configured to compute the partial compliance score by:
computing an ideal nutritional vector comprising ideal average daily intakes of the macronutrients or micronutrients, each of the ideal average daily intakes being computed as an average value between a minimum quantity of intake and a maximum quantity of intake comprised in the values of daily intakes of the user diet pattern,
computing an ideal variance nutritional vector comprising standard deviations from the ideal average daily intakes;
computing a further ideal nutritional vector on the basis of the time of the day at which the computation is performed, the further ideal nutritional vector comprising percentage values of the ideal average daily intakes indicating the percentages of the ideal average daily intakes that could have been consumed at the time of the day by the user;
computing a nutritional intake vector on the basis of the user nutrition track table, the nutritional intake vector comprising intakes of the macronutrients or micronutrients at the time of the day;
for each component of the nutritional intake vector, creating a Gaussian function whose mean value is the corresponding component of the further ideal nutritional vector and whose standard deviation is the corresponding component of the ideal variance nutritional vector, applying each Gaussian function to the respective component of the nutritional intake vector and obtaining a partial score vector comprising individual compliance scores of the macronutrients or micronutrients; and
computing the partial compliance score as a weighted sum of the individual compliance scores.

Preferably, the processing module is further configured to compute the weight value as a weighted sum of individual weight values, each individual weight value indicating the compliance of the target food intake with a respective food rule of the number of food rules in the user diet pattern.

Preferably, the processing module is further configured to compute the individual weight value Wᵣ(j) as:

$${W}_{r} ( j ) = \{ \begin{matrix}F ( t ) , & if FIQ \geq MINQ \\ 1 , & if FIQ ∈ [ MINQ , MAXQ ] \\ {e}^{k ( MAXQ - FIQ )} , & if FIQ > MAXQ\end{matrix}$$

where FIQ is a food intake quantity indicating the food consumed by the user for the food category of the target food at the time of the day, MINQ is a minimum ideal quantity of food to be consumed in accordance with the respective food rule; MAXQ is a maximum ideal quantity of food to be consumed in accordance with the respective food rule, k is a numerical constant comprised in the range [0, 1] and F(t) is a function of time defined as:

$$F ( t ) = \{ \begin{matrix}1 , & if MINQ = 0 \\ 1 - h \cdot {t}^{10 / min} , & if MINQ > 0\end{matrix}$$

where:

$$h = \frac{1}{{duration}^{10 / min}}$$

$$and$$

$$min = \frac{MINQ}{2 \cdot referenceIntake}$$

where duration is a parameter corresponding to the frequency of intake comprised in the respective food rule and referenceIntake is the reference quantity of intake comprised in the respective food rule.

According to embodiments of the present invention, the processing module is further configured to merge the diet patterns of at least two users into a diet pattern union.

Preferably, the processing module is further configured to compute the diet pattern union as the union of the macronutrients or micronutrients or bioactive compounds of the diet patterns of the at least two users, wherein the nutritional information of the macronutrients or micronutrients or bioactive compounds in the diet pattern union is associated with the sum of the corresponding nutritional information in the diet patterns of the at least two users.

Preferably, the processing module is further configured to compute a food rule in the diet pattern union as the union of corresponding food rules in the diet patterns of the at least two users, wherein the corresponding food rules comprise a same food category, a same reference quantity of intake and respective minimum numbers of portions and maximum numbers of portions, by:
transforming the frequencies of intake of the corresponding food rules in a common frequency of intake;
adjusting the minimum number and the maximum number of portions, for each of the corresponding food rules, according to the common frequency;
summing the adjusted minimum numbers of portions; and
summing the adjusted maximum numbers of portions.
   Preferably, the processing module is further configured to receive from the user a list of foods and to compute a further compliance score of the list of foods with the diet pattern.

According to a second aspect, the present invention provides method for supporting a user in the selection of food, the method comprising:
a) determining a diet pattern for the user, the diet pattern comprising nutritional information of a number of macronutrients or micronutrients or bioactive compounds into which food may be decomposed, the diet pattern further comprising a number of food rules, each food rule comprising at least one food category and, for each food category, a frequency of intake and a corresponding quantity of intake; and
b) computing a compliance score of a food indicated by the user with the diet pattern; and
c) notifying the user of the compliance score.

According to a third aspect, the present invention provides a computer program product comprising computer-executable instructions for performing, when the program is run on a computer, the steps of the method set forth above.

### BRIEF DESCRIPTION OF THE DRAWINGS

The present invention will become clearer from the following detailed description, given by way of example and not of limitation, to be read with reference to the accompanying drawings, wherein:

FIG. 1 schematically shows a system for supporting a user in the selection of food according to an embodiment of the present invention;

FIG. 2 is a flow chart illustrating a first use case of application of the present invention;

FIG. 3 is a flow chart illustrating a second use case of application of the present invention; and

FIG. 4 is a flow chart illustrating a third use case of application of the present invention.

### DETAILED DESCRIPTION OF PREFERRED EMBODIMENTS OF THE INVENTION

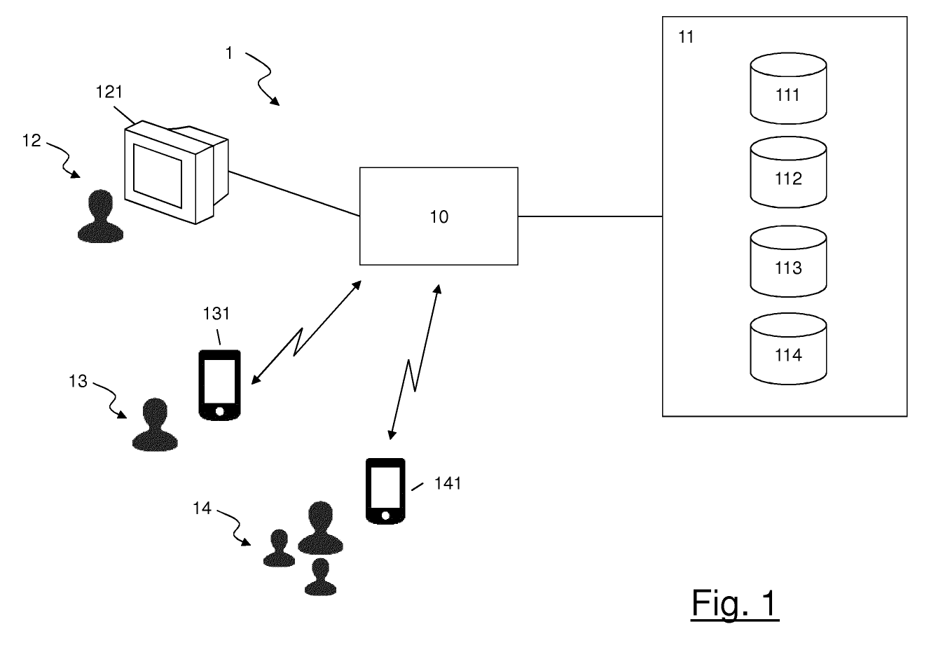

FIG. 1 schematically shows a system 1 for supporting a user in the selection of food according to an embodiment of the present invention.

The system 1 preferably comprises a processing module 10, which may be installed, for instance, on a computer. Moreover, the system 1 preferably comprises a set of databases 11. The databases 11 may be installed onto a memory module of the computer that hosts the processing module 10, or they may be installed onto a remote computer.

The processing module 10 is preferably connected to a number of devices that allow a number of operators and users to access the system 1 and interact with it, as it will be described herein after. In particular, in the exemplary system 1 shown in FIG. 1, the processing module 10 is connected to a first device 121 of a nutritionist 12, to a second device 131 of a user 13, and to a third device 141 of a group of users 14. Each device 121, 131, 141 is preferably connected to the processing device 10 through a data communication network such as the Internet. The connection between each device 121, 131, 141 and the processing module 10 may be a wired or wireless connection. Each device 121, 131, 141 may be a personal computer, a tablet, a smartphone, etc.

The processing module 10 is configured to communicate with operators and users who wish to interact with the system 1, such as for example the nutritionist 12 and the user 13. In particular, the processing module 10 is configured to exchange information and data with the nutritionist 12 and the user 13 though their respective devices 121, 131. The processing module 10 is also configured to receive data from the nutritionist 12 for customizing a dietary model (or diet pattern) for the user 13 and to send to the user 13 a notification of the customized diet pattern, as it will be described in greater detail herein after.

The functionalities provided by the system 1 of the present invention are preferably made available to the nutritionist 12 by means of a web browser running on the nutritionist's device 121, so that the nutritionist 12 may access a web page of a web application residing on the processing module 10. The nutritionist 12 may therefore use the web application for interacting with the components (in particular, the processing module 10) of the system 1. The same functionalities may be made available to the users 13 or groups of users 14 by means of a mobile application running on the users' devices 131, 141. The users 13 or groups of users 14 may therefore use the mobile application for interacting with the components of the system 1 (in particular, the processing module 10).

The processing module 10 is further configured to access the information stored in the databases 11. The databases 11 preferably comprises a diet pattern database 111, a food database 112, a user database 113 and a group database 114.

The diet pattern database 111 preferably comprises a set of pre-determined diet patterns that may be customized by the nutritionist 12 on the basis of the user's needs through the system 1 of the present invention. A diet pattern that is customized for a user 13 according to embodiments of the present invention will be referred to as “user diet pattern”.

According to embodiments of the present invention, a diet pattern contains nutritional information comprising macronutrients and/or micronutrients necessary to satisfy the user's nutritional needs and the respective daily intakes. The nutritional information comprising macronutrients and micronutrients will be referred to also as “nutritional composition” of the diet pattern. According to an embodiment of the present invention, macronutrients and micronutrients are organized in categories as it follows: energy, dietary total fibers, vitamins, water and minerals. The amount of energy may be measured in kcal, the amount of fibers, vitamins, water and minerals in grams. The energy category preferably comprises proteins, carbohydrates and total fats. The total fats may comprise saturated fatty acids and unsaturated fatty acids (monounsaturated and polyunsaturated fatty acids). The vitamins may comprise soluble vitamins, such as thiamin, riboflavin, niacin, pantothenic acid, vitamin B6, biotin, folic acid, vitamin B12, vitamin C, and fat-soluble vitamins, such as vitamin A, vitamin D, and vitamin E. Minerals may comprise calcium, chlorine, iron, iodine, magnesium, phosphorus, potassium, selenium, manganese, zinc, sodium, copper. The amount of vitamins and minerals may be measured in mg or μg. For each macronutrient and micronutrient, the diet pattern comprises a daily reference quantity of intake and/or a range for the daily quantity of intake (comprising a minimum quantity of intake and a maximum quantity of intake) and a unity of measure (e.g. grams, mg or μg).

Moreover, the diet pattern preferably comprises a number of food rules. Each food rule preferably comprises one or more categories of food, the frequency of intake (e.g. daily, weekly, monthly), a minimum number of portions and a maximum number of portions for the considered food categories and, for each food category, a reference quantity of intake. The food category may be one of the following: fresh fruit, dried fruit, vegetables, salad, bread, pasta, rice, biscuits, cereals, potatoes, meat, fish, eggs, cold cuts, fresh legumes, dried legumes, milk, soy milk, yogurt, seasoned cheese, fresh cheese, butter, oil, wine, beer, sugar, honey, cakes, jam, tofu or others soy products, other meats.

The diet pattern may optionally comprise further nutritional information related to a set of bioactive compounds, wherein the bioactive compounds comprise nutritional compounds that help preventing and/or treating diseases and disorders. Bioactive compounds may comprise: omega-3, omega-6, ascorbic acid, carotenoid, isoflavones, polyphenols, quercetin, resveratrol, lycopene, folic acid, lutein, fiber, beta-glucan. The diet pattern specifies, for each bioactive compound, the necessary daily quantity to reach the therapeutic target. For each bioactive compound, the diet pattern preferably comprises a minimum quantity of intake and a maximum quantity of intake and a unity of measure (e.g. grams, mg or μg).

According to the present invention, a user diet pattern is a diet pattern that has been customized to meet a user's profile. It preferably contains daily quantities of intake (minimum, maximum and reference quantities of intake) of macronutrients, micronutrients and bioactive compounds which are tailored to the user's needs and habits. This customization, as already mentioned above, may be performed by the nutritionist 12 through the system 1 of the present invention. Furthermore, a user diet pattern may comprise information about intolerances and/or allergies of the user, i.e. a list of foods to which the user is allergic or intolerant.

In Table 1 herein below, an example of a diet pattern will be given. The exemplary diet pattern that will be described is a diet pattern which refers to the so-called “Mediterranean diet”. In table 1, the expression “Nutritional composition” indicates the nutritional information detailing macronutrients and micronutrients. The abbreviations “Ref”, “Max” and “Min” in Table 1 will refer to, respectively, a reference quantity of intake, a maximum quantity of intake and a minimum quantity of intake. The term “Unit” will refer to the unit of measure. The abbreviations “Sat” and “Unsat”, as related to the Fat macronutrient, will refer to, respectively, saturated fatty acids and unsaturated fatty acids. The abbreviations “Mono” and “Poly”, as related to unsaturated fatty acids, will refer to, respectively, monounsaturated fatty acids and polyunsaturated fatty acids. In the “Food rules” section of the diet pattern, the abbreviations “Max” and “Min” will refer to a maximum number of portions and a minimum number of portions, respectively, the abbreviation “Freq” will refer to a frequency of intake of the considered food, and the abbreviation “Ref” will refer to a reference quantity of intake per portion.

TABLE 1
Mediterranean diet pattern

| Nutritional composition: | | | | Ref | Max | Min | Unit |
|---|---|---|---|---|---|---|---|
| Energy | | | | 2000 | | | Kcal |
| | Protein | | | | 91.4 | 54.1 | g |
| | Carbohydrates | | | | 309.1 | 282.8 | g |
| | Fat | Sat | | | 16.8 | 11.7 | g |
| | Fat | Unsat | Mono | | 34.7 | 26.2 | g |
| | Fat | | Poly | | 10.4 | 7.0 | g |

| Dietary Total Fibers | | | 56.0 | 33.0 | g |
|---|---|---|---|---|---|
| Vitamins | Soluble | Thiamin | 3.7 | 1.7 | mg |
| | | Riboflavin | 1.7 | 1.3 | mg |
| | | Niacin | 29.1 | 14.6 | mg |
| | | Pantothenic | 3.4 | 1.5 | mg |
| | | acid | | | |
| | | Vitamin B6 | 4.3 | 2.7 | mg |
| | | Biotin | 22.0 | 9.6 | μg |
| | | Folic acid | 404.8 | 236.0 | μg |
| | | Vitamin B12 | 6.7 | 1.9 | μg |
| | | Vitamin C | 174.4 | 77.3 | mg |
| | Fatsoluble | Vitamin A | 652.8 | 150.7 | μg |
| | | Vitamin D | 3.6 | 0.6 | μg |
| | | Vitamin E | 20.6 | 15.7 | mg |

| Water | | 2561.4 | 2293.4 | g |
|---|---|---|---|---|
| Sugars | | 101.30 | 90.50 | g |
| Alcohol | | 10 | 0 | g |
| Minerals | Calcium | 815.6 | 568.4 | mg |
| | Chlorine | 1226.4 | 316.1 | mg |
| | Iron | 18.01 | 14.0 | mg |
| | Iodine | 152.1 | 58.0 | μg |
| | Magnesium | 148.0 | 70.05 | mg |
| | Phosphorus | 2283.2 | 1265.3 | mg |
| | Potassium | 4024.3 | 2810.5 | mg |
| | Selenium | 89.2 | 19.0 | μg |
| | Manganese | 0.2 | 0.017 | mg |
| | Zinc | 17.7 | 8.7 | mg |
| | Sodium | 3022.7 | 1479.7 | mg |
| | Copper | 1.2 | 0.1 | mg |

| Bioactive compounds: | Min | Max | Unit |
|---|---|---|---|
| Omega3 | 2 | 4 | g |
| Ascorbic acid | 60 | 90 | mg |
| Carotenoid | 6 | 18 | mg |
| Isoflavones | 60 | 80 | mg |
| Polyphenols | 1 | 3 | g |
| Quercetin | 50 | 300 | mg |
| Resveratrol | 200 | 400 | mg |
| Lycopene | 1 | 15 | mg |
| Folic acid | 0.2 | 0.4 | mg |
| Lutein | 6 | 20 | mg |
| Fiber | 25 | 30 | g |

Food rules:

| Min | Max | Freq | Food | Ref | Unit |
|---|---|---|---|---|---|
| 2 | 2 | daily | Vegetables | 250 | g |
| | | | Salad | 50 | g |
| 3 | 3 | daily | Fresh fruit | 150 | g |
| | | | Dry fruit | 30 | g |
| 2 | 3 | daily | Bread | 50 | g |
| 1 | 1 | daily | Pasta | 80 | g |
| | | | Rice | 80 | g |
| 1 | 1 | daily | Biscuits | 20 | g |
| 1 | 2 | weekly | Potatoes | 200 | g |
| 3 | 5 | weekly | Meat | 100 | g |
| 2 | 3 | weekly | Fish | 150 | g |
| 1 | 2 | weekly | Eggs | 60 | g |
| 0 | 3 | weekly | Cold cuts | 50 | g |
| 2 | 2 | weekly | Fresh legumes | 100 | g |
| | | | Dry legumes | 30 | g |
| 14 | 14 | weekly | Milk | 125 | g |
| | | | Yogurt | 125 | g |
| 0 | 4 | weekly | Seasoned cheese | 50 | g |
| | | | Fresh cheese | 100 | g |
| 0 | 5 | weekly | Butter | 10 | g |
| 2 | 3 | daily | Oil | 10 | g |
| 0 | 1 | daily | Wine | 100 | g |
| | | | Beer | 100 | g |
| 0 | 3 | daily | Sugar | 5 | g |
| | | | Honey | 5 | g |
| 0 | 1 | weekly | Cakes | 100 | g |
| 0 | 3 | weekly | Jam | 7 | g |
| 0 | 3 | daily | Soy milk | 125 | g |

Furthermore, in Table 2 herein below, another example of a diet pattern will be given, which refers to the so-called “Dash diet” suitable for hypertensive patients.

TABLE 2
Dash diet pattern

| Nutritional composition: | | | | Ref | Max | Min | Unit |
|---|---|---|---|---|---|---|---|
| Energy | | | | 2000 | | | Kcal |
| | Protein | | | | 94 | 77 | g |
| | Carbohydrates | | | | 247.73 | 202.69 | g |
| | Fat | Sat | | | 23.23 | 19.00 | g |
| | Fat | unsat | mono | | 46.24 | 37.83 | g |
| | Fat | unsat | poly | | 22.52 | 18.43 | g |

| Dietary Total Fibres | | | 42.75 | 34.97 | g |
|---|---|---|---|---|---|
| Vitamins | Soluble | Thiamin | 1.96 | 1.60 | mg |
| | | Riboflavin | 2.72 | 2.22 | mg |
| | | Niacin | 25.43 | 20.80 | mg |
| | | Pantothenic | 3.07 | 2.51 | mg |
| | | acid | | | |
| | | Vitamin B6 | 3.12 | 2.56 | mg |
| | | Biotin | 34.21 | 27.99 | μg |
| | | Folic acid | 555.88 | 454.81 | μg |
| | | Vitamin B12 | 2.64 | 2.16 | μg |
| | | Vitamin C | 243.21 | 198.99 | mg |
| | Fatsoluble | Vitamin A | 2367.53 | 1937.07 | μg |
| | | Vitamin D | 6.49 | 5.31 | μg |
| | | Vitamin E | 32.24 | 26.38 | mg |

| Water | | 2919.09 | 2388.35 | g |
|---|---|---|---|---|
| Sugars | | 131.54 | 107.63 | g |
| Alcohol | | 0 | 0 | g |
| Minerals | Calcium | 1567.00 | 1282.09 | mg |
| | Chlorine | 1521.85 | 1245.15 | mg |
| | Iron | 19.96 | 16.33 | mg |
| | Iodine | 221.48 | 181.21 | μg |
| | Magnesium | 159.77 | 294.36 | mg |
| | Phosphorus | 2354 | 1926 | mg |
| | Potassium | 5512.43 | 4510.07 | mg |
| | Selenium | 31.29 | 25.6 | μg |
| | Manganese | 3.56 | 2.91 | mg |
| | Zinc | 18.39 | 15.05 | mg |
| | Sodium | 1948.48 | 1594.21 | mg |
| | Copper | 1.42 | 1.6 | mg |

| Bioactive compounds: | Min | Max | Unit |
|---|---|---|---|
| Omega3 | 2 | 4 | g |
| Ascorbic acid | 60 | 90 | mg |
| Carotenoid | 6 | 18 | mg |
| Isoflavones | 60 | 80 | mg |
| Polyphenols | 1 | 3 | g |
| Quercetin | 50 | 300 | mg |
| Resveratrol | 200 | 400 | mg |
| Lycopene | 1 | 15 | mg |
| Folic acid | 0.2 | 0.4 | mg |
| Lutein | 6 | 20 | mg |
| Fiber | 25 | 30 | g |

Food rules:

| Min | Max | Freq | Food | Ref | Unit |
|---|---|---|---|---|---|
| 7 | 8 | daily | Cereals | 30 | g |
| | | | Bread | 30 | g |
| | | | Pasta | 65 | g |
| | | | Rice | 65 | g |
| 4 | 5 | daily | Vegetables | 250 | g |
| 4 | 5 | daily | Fresh fruit | 150 | g |
| 0 | 6 | daily | Meat | 30 | g |
| | | | Fish | 30 | g |
| | | | Eggs | 60 | g |
| 4 | 5 | weekly | Dry fruits | 30 | g |
| | | | Fresh legumes | 65 | g |
| 2 | 3 | daily | Oil | 15 | g |
| 0 | 5 | weekly | Sugar | 15 | g |
| | | | Jam | 15 | g |

As mentioned above, according to embodiments of the present invention, the diet pattern may be personalized for a user by the nutritionist 12 though the system 1 of the present invention on the basis of a user's profile comprising information such as gender, height, weight, BMI (Body Mass Index), age, diseases, intolerances, allergies and user's needs and/or habits and/or objectives, such as calories expenditure. As already mentioned before, according to the present invention, the user 13 may perform a food choice on the basis of her/his preferences, and the system provides a compliance feedback on the basis of the diet pattern. Details will be given in the following description.

The food database 112 preferably comprises a number of entries relating to foods and corresponding nutritional information. The foods may comprise “elementary” foods (such as, for instance, apple, egg, milk, carrot, etc.) and more elaborated foods derived from application of recipes. Each food is preferably associated with a food name, a food category and the corresponding nutritional information, which may comprise a quantity (e.g. in grams) of a number of macronutrients and/or micronutrients and/or bioactive compounds, such as those listed in the exemplary entry of the food database shown in Table 3 herein after. In such table, the first line comprises the food name, the second line the food category and the following lines the nutritional information related to a given reference quantity of the food (e.g. 100 g in Table 3).

TABLE 3

| | |
|---|---|
| Food name | Bread |
| Food category | Bread |
| Reference quantity | 100 g |
| Water (g) | 31 |
| Protein (g) | 8.1 |
| Total fat (g) | 0.5 |
| Carbohydrates (g) | 63.5 |
| Sugars (g) | 2 |
| Dietary Total Fibre (g) | 3.8 |
| Alcohol (g) | 0 |
| Energy (kcal) | 275 |
| Sodium (mg) | 293 |
| Potassium (mg) | |
| Iron (mg) | 0.7 |
| Calcium (mg) | 17 |
| Phosphorus (mg) | 77 |
| Magnesium (mg) | |
| Zinc (mg) | |
| Copper (mg) | |
| Selenium (μg) | |
| Thiamin (mg) | 0.06 |
| Riboflavin (mg) | 0.06 |
| Niacin (mg) | 0.8 |
| Vitamin A (μg) | 0 |
| Vitamin C (mg) | 0 |
| Vitamin E (mg) | |

The user database 113 preferably comprises information related to users 13. In particular the user database 113 comprises, for each user 13:
a user's profile, comprising data identifying the user, such as gender, height, weight, BMI, age, diseases, allergies;
a food diary, comprising information about the foods consumed by the user subdivided in food categories (e.g. fruit, bread, vegetable, meat). The food diary is associated with a food diary table that preferably tracks what the user 13 has consumed, how many portions and the quantity of each portion. In particular, the food diary table comprises, for each consumed food, a numerical identifier ID, a date and time of the food intake (i.e. a “timestamp”), a food category, the quantity of the consumed food (in grams or number of portions and quantity, in grams, per portion), and a user identifier. An exemplary food diary table is shown in Table 4 herein after. It is assumed that the labels of the table columns are self explaining.

TABLE 4

| ID | Timestamp | Food Category | Portions | Quantity [g] | User |
|---|---|---|---|---|---|
| 01 | 2016-02-08 | Bread | 1 | 60 | 02 |
| | 08:30 | | | | |
| 02 | 2016-02-08 | Bread | 1 | 60 | 02 |
| | 12:30 | | | | |

a user nutrition track table, comprising detailed nutritional information about each food consumed by the user. The nutritional information is preferably expressed as values of a set of macronutrients and/or micronutrients and/or bioactive compounds composing the consumed food. These values represent the quantity of each macronutrient or micronutrient or bioactive compound in the food (e.g. in Kcal, grams, mg or μM. The macronutrients and/or micronutrients and/or bioactive compounds are those described above for the diet pattern. The table comprises, for each food consumed by the user, a numerical identifier, a date and time of the food intake (the timestamp), a name indicating the food, the nutritional values and a user identifier. An exemplary nutrition track table is shown in Table 5 herein after, where it is assumed that the labels of the table columns are self explaining. For the sake of simplicity, only energy and protein have been reported in Table 5 as nutritional composition.

TABLE 5

| | | | Nutritional values | | | |
|---|---|---|---|---|---|---|
| ID | Timestamp | Food | Energy | Protein | . . . | User |

| | | | | | |
|---|---|---|---|---|---|
| 01 | 2016-02-08 | Apple | 67.5 | 0.3 | 02 |
| | 08:30 | | | | |
| 02 | 2016-02-08 | Yogurt | 45 | 4.125 | 02 |
| | 08:30 | | | | |

a diet pattern of the user, customized on the basis of the user's profile.

The group database 114 preferably comprises information related to groups of users 14, such as families, sport teams or other groups. In particular, the group database 114 comprises, for each group 14:
the diet pattern of each user of the group;
a diet pattern union, which is calculated on the basis of the diet patterns of the users of the group, as it will be described in detail herein after;
a food list, comprising, for instance, foods purchased for the whole group. The food list is associated with a food list table that may comprise information about the foods entered in the food list such as: a numerical identifier of the food list entry, a food category, the quantity of the purchased food (in grams or number of portions and quantity, in grams, per portion), and a group identifier. An exemplary food list table is shown in Table 6 herein after;

TABLE 6

| ID | Food Category | Portions | Quantity (g) | Group |
|---|---|---|---|---|
| 01 | Bread | 4 | 60 | 02 |
| 02 | Vegetables | 5 | 60 | 02 |

a group nutrition track table, comprising detailed nutritional information about each food in the food list (e.g. energy, protein, carbohydrates, dietary fiber, vitamins, etc.). The nutritional information is preferably expressed as values of a set of macronutrients and/or micronutrients and/or bioactive compounds composing the food. These values represent the quantity of each nutrient in the food (e.g. in Kcal, grams, mg or μM. The group nutrition track table may comprise, for each food in the food list, a numerical identifier of the food entry, a food name, the nutritional values and a group identifier. An exemplary group nutrition track table is represented in Table 7 herein below.

TABLE 7

| | | Nutritional values Food ID Group | | | |
|---|---|---|---|---|---|
| ID | Food | Energy | Protein | . . . | Group |
| 01 | Apple | 67.5 | 0.3 | | 02 |
| 02 | Yogurt | 45 | 4.125 | | 02 |

As mentioned above, the processing module 10 stores the information provided by the user 13 or the group of users 14 in the user database 113 and the group database 114, respectively. The food diary table and the nutrition track table in the user database 113 are preferably updated each time the user 13 inserts a food in the system 1, for instance a consumed food or a food that she/he wishes to purchase. The food list table and the group nutrition track table in the group database 114 are preferably updated each time an item is added to the food list.

Further, the processing module 10 is preferably configured to execute a set of algorithms comprising:
a compliance score algorithm, which evaluates the compliance of a food with a diet pattern given in input, and computes a corresponding score;
a diet pattern union algorithm, which, given in input two or more diet patterns, determines their diet pattern union; and
a food list compliance algorithm for checking the compliance of a number of foods (e.g. the foods in a food list) with a diet pattern (either a user diet pattern or a diet pattern union) given in input to the algorithm.

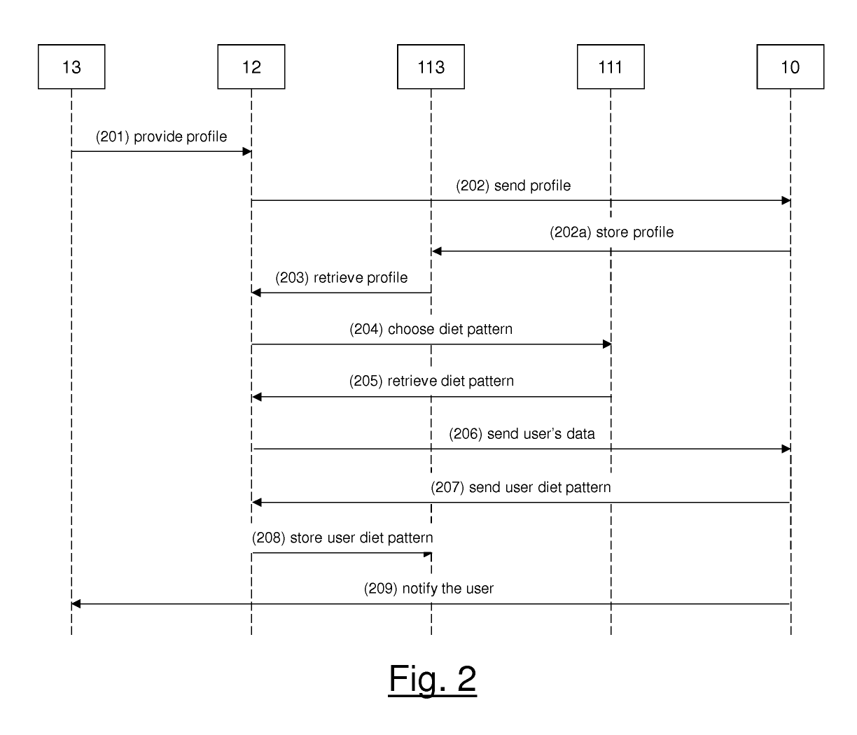

FIG. 2 is a flow chart illustrating a first use case of application of the present invention. This use case relates to the situation according to which the nutritionist 12 customizes a diet pattern for a user 13.

It is to be noticed that the transmission of data from the nutritionist 12 (namely from the nutritionist's device 121) to the databases 113, 111, and vice versa, is preferably performed through the processing module 10. In the following description, reference to the processing module 10 as intermediate device between the nutritionist's device 121 or the user's device 131 and the databases may be omitted, for simplicity.

At step 201 of the flowchart of FIG. 2, the user 13 provides the nutritionist 12 with the data of her/his profile, comprising gender, height, weight, BMI, age, diseases, allergies. In an embodiment of the present invention the user 13 can supply these data by filling-in a pre-determined questionnaire available through the mobile application installed on the user's device 131.

After receiving the user's data from the user's device 131, the nutritionist 12 preferably sends them to the processing module 10 through her/his device 121 (step 202), then the processing module 10 forwards the data to the user database 113 so as to store them on it (step 202a).

The operations described above may be performed once, for instance upon installation on the user's device 131 of the mobile application providing the functionalities of the system 1. Subsequently, the user 13 may provide the nutritionist 12 with updates or changes of her/his data in the same manner as described herein above. Alternatively, the user 13 may provide the the data of her/his profile to the processing module 10 directly, e.g. by means of the mobile application cited above.

To determine a customized diet pattern for a given user 13, at step 203, the nutritionist 12 preferably retrieves the user's profile from the user database 113. Then, on the basis of the data of such profile, the nutritionist 12 preferably accesses the diet pattern database 111 and looks for a diet pattern, among the pre-determined diet patterns that are stored in the diet pattern database 111, which meets the user's profile, according to the nutritionist's experience. Then, the nutritionist 12 chooses the diet pattern (step 204) for the user 13 and retrieves it from the diet pattern database 111 (step 205). Once the chosen diet pattern has been retrieved from the diet pattern database 111, the nutritionist 12 preferably interacts with the processing module 10 in order to customize the chosen diet pattern for the user. Customization is performed on the basis of the user's profile which, optionally, may comprise also information that the user may provide to the nutritionist 12 such as user's food preferences and user's needs or habits (e.g. a user's degree of physical activity). For instance, the nutritionist 12, through her/his device 121, provides the processing module 10 with the user's profile and other information about the user such as physical activity degree, food not appreciated, intolerances (step 206). This may be performed, alternatively, by the user 13 through her/his device 131. The processing module 10, on the basis of the user's data, computes a user's energy (or calories) requirement and updates the chosen diet pattern on the basis of the computed energy requirement in order to get the user diet pattern. This computation may be performed on the basis of, for instance, the known Harris-Benedict equations that provide an estimate of an individual's basal metabolic rate (BMR) and daily kilocalorie requirements. The user diet pattern so obtained is then sent to the nutritionist 12 (step 207).

At step 208 the nutritionist 12 preferably associates the user diet pattern with the user 13 and sends the user diet pattern to the user database 113 so as to store it. Then, at step 209, the processing module 10 preferably notifies the user 13 with the user diet pattern, for instance through the mobile application installed on the user's device 131.

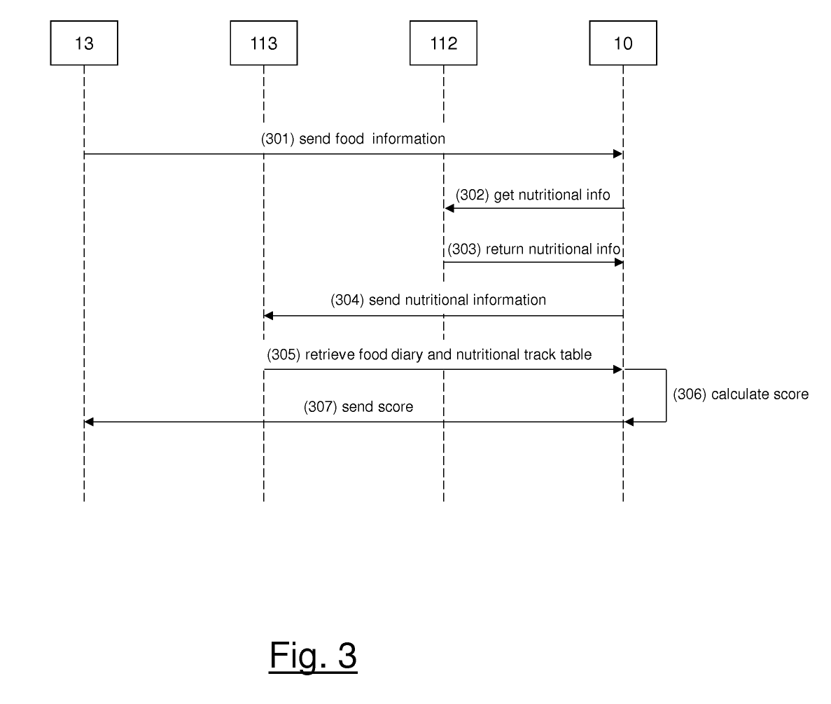

FIG. 3 is a flow chart illustrating a second use case of application of the present invention. According to this second use case, the user 13 stores in the system 1 information about a food that she/he has consumed or is going to consume (in the present description and in the claims, it will be referred to also as “target food”), and the relevant food intake, and receives a compliance score from the system 1.

At step 301, the user 13 preferably sends to the processing module 10 information about the food (name of the food and food intake). At step 302, the processing module 10, on the basis of the food name received from the user 13, accesses the food database 112 to get detailed nutritional information about the food and to get the food category. At step 303, the food database 112 returns the nutritional information and the food category to the processing module 10. Then, at step 304, the processing module 10, starting from the nutritional information of the reference quantity of the considered food, computes the nutritional information related to the food as indicated by the user 13 and accesses the user database 113 to update the food diary table and the user nutrition track table.

For instance, at step 301, the user 13 may specify that she/he has eaten 2 portions of bread of 60 g each. The processing module 10 retrieves from the food database 112 the nutritional information related to a reference quantity of bread, namely 100 g (steps 302 and 303). The, the processing module 10, starting from the nutritional information of the reference quantity of bread, computes the nutritional information related to the portions of bread eaten by the user 13 and updates the food diary table and the user nutrition track table with the computed nutritional information (step 304). This is exemplarily represented in the food diary table of Table 4 herein above.

At step 305, the processing module 10 preferably retrieves the updated food diary table and user nutritional track table and, on the basis of the data contained therein, calculates the compliance score of the food with the user diet pattern (step 306). This computation is performed by applying the compliance score algorithm that will be described in detail herein after.

According to the present invention, the compliance score algorithm evaluates the compliance of a food (and of the relevant food intake) with a specific user diet pattern. The compliance of a food with a user diet pattern is preferably evaluated taking into account the relevant food intake and the food previously consumed by the user and tracked in the food diary and in the nutrition track table. The algorithm preferably calculates a compliance score as an integer number within a given compliance range. The compliance score may be for instance comprised between 0 and 100. The minimum value of the compliance score, e.g. 0, is returned by the algorithm for a food that is not compliant with a user diet pattern (e.g. this is the compliance score which is returned in case the user is allergic to the considered food). The maximum value of the compliance score, e.g. 100, is returned by the algorithm for a food that is completely compliant with a user diet pattern.

The compliance score is calculated as follows:

Sₜ=Sₙ×Wᵣ [1]

where Sₜ is the compliance score returned by the algorithm, Sₙ is a partial compliance score indicating the compliance of the considered food with respect to the nutritional composition and the bioactive compounds specified in the user diet pattern and assuming a value within the compliance range, and Wᵣ is a weight value which indicates whether the food rules comprised in the user diet pattern are fulfilled or not, as it will be described in greater detail herein after. The weight Wᵣ may preferably assume a value comprised between 0 an 1.

In the following lines, the computation of the partial compliance score Sₙ will be described first.

The processing module 10, starting from the user diet pattern, preferably generates an ideal nutritional vector INVμ containing a number of numerical values indicating the ideal average daily intakes of the micronutrients and macronutrients comprised in the user diet pattern. The ideal average daily intake of a nutrient is preferably computed by the processing module 10 as the average value between the maximum quantity of intake and the minimum quantity of intake specified in the user diet pattern. For instance, the ideal nutritional vector INVμ, may be represented as follows:
INVμ=[energy_{μ}, protein_{μ}, carbohydrate_{μ}, saturatedFattyAcids_{μ}, monounsaturated_{μ}, polyunsatu rated_{μ}, d ietaryTotal Fibre_{μ}, thiamin_{μ}, riboflavin_{μ}, niacin_{μ}, pantothenicAcid_{μ}, vitamin B6_{μ}, biotin_{μ}, folicAcid_{μ}, vitamin B12_{μ}, vitaminC_{μ}, vitaminA_{μ}, vitaminD_{μ}, vitaminE_{μ}, water_{μ}, sugars_{μ}, alcohol_{μ}, calcium_{μ}, chlorine_{μ}, iron_{μ}, iodine_{μ}, magnesium_{μ}, phosphorus_{μ}, potassium_{μ}, selenium_{μ}, manganese_{μ}, zinc_{μ}, sodium_{μ}, copper_{μ}, ascorbicAcid_{μ}, carotenoid₉₈₂]
   wherein each component of the vector indicates the ideal average value of daily intake of the corresponding macronutrient or micronutrient, calculated using the maximum quantity of intake and the minimum quantity of intake, reported in the diet pattern (see Table 1 and Table 2 above for exemplary diet patterns). For instance, the notation “energy_{μ}” indicates the ideal average value of daily energy intake, the notation “protein_{μ}” indicates the ideal average value of daily protein intake, and so on.

A numerical example of an ideal nutritional vector INVμ calculated for the diet pattern of the “Mediterranean diet” (Table 1) is as follows:
INVμ=[2000, 72.75, 295.95, 14.25, 30.45, 8.7, 44.5, 2.7, 1.5, 16.85, 2.45, 3.5, 15.8, 320.4, 4.3, 125.85, 401.75, 2.1, 18.15, 2427.4, 95.9, 5, 692, 771.25, 16.01, 105.05, 109.03, 1774.25, 3417.4, 54.1, 0.11, 13.2, 2251.2, 0.65, 75, 12].
   Moreover, the processing module 10 preferably generates an ideal variance nutritional vector INVσ containing a number of numerical values indicating the standard deviations from the ideal average daily intakes specified in the ideal nutritional vector INVμ. The standard deviation for a nutrient is preferably computed by the processing module 10 as the deviation from the average value computed between the maximum quantity of intake and the minimum quantity of intake specified in the user diet pattern. If the user diet pattern contains a reference value of intake, a fixed standard deviation from the average value is considered, for instance 5%.

For instance, the ideal variance nutritional vector INVσ, may be represented as follows:
INVσ=[energy_{σ}, protein_{σ}, carbohydrate_{σ}, saturatedFattyAcids_{σ}, monounsaturated_{σ}, polyunsaturated_{σ}, dietaryTotalFibre_{σ}, thiamin_{σ}, riboflavin_{σ}, niacin_{σ}, pantothenicAcid_{σ}, vitamin B6_{σ}, biotin_{σ}, folicAcid_{σ}, vitamin B12_{σ}, vitaminC_{σ}, vitaminA_{σ}, vitaminD_{σ}, vitaminE_{σ}, water_{σ}, sugars_{σ}, alcohol_{σ}, calcium_{σ}, chlorine_{σ}, iron_{σ}, iodine_{σ}, magnesium_{σ}, phosphorus_{σ}, potassium_{σ}, selenium_{σ}, manganese_{σ}, zinc_{σ}, sodium_{σ}, copper_{σ}, ascorbicAcid_{σ}, carotenoid_{σ}]
   wherein the notation “energy,” indicates the ideal variance of the daily energy intake and the other terms indicate the ideal variances of daily intakes for the other micronutrients and macronutrients listed above. A numerical example of an ideal variance nutritional vector INVσ calculated for the diet pattern of the “Mediterranean diet” is as follows:
INVσ=[100, 18.65, 13.15, 2.55, 4.25, 1.7, 11.5, 1, 0.2, 12.25, 0.95, 0.8, 6.2, 85.4, 2.4, 48.55, 251.05, 1.5, 2.45, 134, 5.4, 5, 123.6, 455.15, 2.005, 47.05, 38.975, 508.95, 606.9, 35.1, 0.0915, 4.5, 771.5, 0.55, 15, 6].
   Then, on the basis of the time of the day at which the system 1 evaluates the compliance of the considered food with the user diet pattern, the processing module 10 preferably computes a further ideal nutritional vector which takes into account that, at that specific time of the day, the user could have consumed only a part of a daily average intake of each macronutrient and micronutrient specified in the ideal nutritional vector INVμ. For instance, at lunch time, the processing module 10 preferably computes an ideal nutritional vector at lunch time INVμL. This further ideal nutritional vector is preferably computed using a percentage matrix PM which comprises percentage values indicating the percentage of the ideal average daily intakes of macronutrients and/or micronutrients that the user could progressively consume at a number of different timeslots during the day, from breakfast to dinner. For each nutrient, the percentage value at dinner shall be equal to 100, indicating that the daily average intake of a nutrient could be entirely consumed at the end of the day. For instance, the percentage matrix PM may comprise the percentage values of the ideal average daily intakes of nutrients at 5 timeslots corresponding to: breakfast time, (e.g. from 00:00 to 09:30), morning snack time (e.g. from 09:31 to 11:00), lunch time (e.g. from 11:00 to 14:00), afternoon snack time (e.g. from 14:00 to 17:00) and dinner time (e.g. from 17:00 to 24:00). An exemplary percentage matrix PM for the nutrients specified in the ideal nutritional vector INVμ above is illustrated in Table 8 herein after. In Table 8, the first column reports the indication of the timeslot and the second column contains the percentage values of the macronutrients and micronutrients.

TABLE 8

| Timeslot | Percentage values |
|---|---|
| Breakfast | [15.17, 10.56, 16.6, 37.91, 17.04, 33.75, 7.98, 10.66, |
| (00:00-09:30) | 10.86, 10.63, 0, 9.21, 21.44, 6.49, 9.97, 9.2, 10.02, |
| | 6.98, 9.31, 0, 8.56, 11.65, 20.13, 21.52, 6.43, 19.94, |
| | 13.39, 14.1, 12.69, 10.01, 12.08, 8.91, 0, 16.73, 27.12, |
| | 5.72, 2.4, 17.98] |
| Snack | [17.42, 16.56, 18.22, 43.41, 19.14, 38.15, 8.83, 11.2, |
| (9:31-11:00) | 10.86, 15.4, 0, 10.21, 33.36, 6.49, 11.37, 12.89, 16.15, |
| | 9.47, 9.38, 0, 17.12, 12.27, 52.57, 50.86, 7.17, 34.35, |
| | 18.29, 21.94, 20, 16.37, 32.95, 14.48, 0, 86.98, 67.8, |
| | 5.72, 2.61, 19.27, 56.757, 56.757] |
| Lunch | [56, 73, 54, 57, 37, 54, 61, 86, 86, 83, 98, 65, 79, 77, |
| (11:00-14:00) | 86, 67, 95, 90, 57, 50, 50, 100, 70, 82, 80, 97, 65, 69, |
| | 72, 41, 98, 71, 55, 61, 67, 95] |
| Snack | [62.82, 77.08, 59.71, 75.42, 55.48, 63.32, 46.24, |
| (14:00-17:00) | 65.44, 69.94, 59.63, 100, 94.9, 87.75, 83.08, 66.78, |
| | 66.87, 79.7, 95.1, 63.54, 100, 85.62, 82.96, 79.42, |
| | 98.33, 85.41, 73.05, 69.64, 74.95, 75.5, 76.34, 80.4, |
| | 67.93, 98.36, 96.79, 81.9, 61.1, 97.43, 70.88, 100, |
| | 100] |
| Dinner | [100, 100, 100, 100, 100, 100, 100, 100, 100, 100, 100, |
| (17:00-24:00) | 100, 100, 100, 100, 100, 100, 100, 100, 100, 100, 100, |
| | 100, 100, 100, 100, 100, 100, 100, 100, 100, 100, 100, |
| | 100, 100, 100] |

The ideal nutritional vector at lunch time INVμL as computed starting from the ideal nutritional vector INVμ reported above is as follows:
INVμL=[1120.00, 53.11, 159.81, 8.12, 11.27, 4.70, 27.15, 2.32, 1.29, 13.99, 2.40, 2.28, 12.48, 246.71, 3.70, 84.32, 381.66, 1.89, 10.35, 1213.70, 47.95, 5.00, 484.40, 632.43, 12.81, 101.90, 70.87, 1224.23, 2460.53, 22.18, 0.11, 9.37, 1238.16, 0.40, 50.25, 11.40].
   The standard deviations from the ideal average daily intakes are preferably assumed to be the same during all the day. Therefore, the ideal variance nutritional vector INVσ may be used to evaluate the compliance at the different timeslots.

In the following description, the computation of the compliance score will be described with reference to the ideal nutritional vector at lunch time INVμL (in particular, the ideal nutritional vector at lunch time INVμL with the exemplary numerical values reported above). This is merely an example as the same computation may be performed when considering any timeslot of the day starting from the corresponding ideal nutritional vector computed for the considered timeslot.

The processing module 10 preferably retrieves from the user database 113 the nutrition track table of the user and uses the data contained therein to calculate a nutritional intake vector at lunch time NIV_{L}. The nutritional intake vector at lunch time NIV_{L} preferably comprises the intakes of macronutrients and micronutrients as stored in the user database 113 (in particular, in the user nutrition track table) at a specific date and time, i.e. the lunch time for the considered example. It is assumed for sake of example that the nutritional intake vector at lunch time NIV_{L} in the example is as follows:
NIV_{L}=[1306.5, 45.1, 214.58, 14.931, 14.454, 5.8325, 23.46, 2.012, 1.3545, 11.905, 1.1615, 2.588, 7.63, 206.05, 1.52, 46.75, 3999.35, 0.097, 6.8465, 1063.525, 77.64, 0, 675.1, 800.3, 10.145, 99.9, 70.25, 906.4, 1658.55, 5.05, 0.0175, 9.94, 993.4, 0.101, 46.75, 3999.35].
   The processing module 10 preferably determines a Gaussian function for each component of the nutritional intake vector at lunch time NIV_{L}, wherein the mean value μ and the standard deviation σ of each Gaussian function are the respective components of the ideal nutritional vector at lunch time INVμ and the ideal variance nutritional vector INVσ. The Gaussian function may be defined according to the following equation:

$$f ( x ) = exp ( - \frac{{( x - μ )}^{2}}{2 {σ}^{2}} ) \qquad [ 2 ]$$

where μ is the mean value and σ is the standard deviation.

Then, the processing module 10 preferably applies to each component of the nutritional intake vector at lunch time NIV_{L} the respective Gaussian function and determines a partial score vector PSV. The partial score vector PSV for the exemplary nutritional intake vector at lunch time NIV_{L} reported above is as follows:
PSV=[82.43, 98.98, 38.15, 67.30, 96.92, 97.56, 99.43, 99.47, 99.42, 99.84, 90.98, 99.15, 96.65, 98.72, 95.53, 96.73, 0.00, 92.37, 89.29, 93.26, 18.65, 94.60, 87.61, 99.25, 90.66, 99.99, 100.00, 97.86, 90.75, 98.69, 94.73, 99.91, 99.44, 98.41, 99.70, 0.00].
   Each component of the partial score vector PSV is a compliance score related to the specific macronutrient or micronutrient comprised in the user diet pattern as listed in the ideal nutritional vector INVμ and the ideal variance nutritional vector INVσ.

The partial compliance score Sₙ of the considered food is preferably computed by the processing module 10 by performing a weighted average of the individual compliance scores comprised within the partial score vector PSV, according to the equation herein below:

$${S}_{n} = \frac{{\sum }_{i = 1}^{N} WGT ( i ) \times PSV ( i )}{{\sum }_{i = 1}^{N} WGT ( i )} \qquad [ 3 ]$$

wherein WGT is a vector of weights, PSV is the partial score vector and N is the number of components of the partial score vector PSV. The weight vector WGT may be equal, for example, to:
WGT=[2, 3, 2, 2, 2, 2, 2, 2, 2, 2, 2, 2, 2, 2, 2, 2, 2, 2, 2, 2, 2, 2, 4, 2, 4, 2, 2, 2, 5, 2, 2, 2, 5, 2, 2, 2].
   Each component of the weight vector WGT preferably corresponds to a specific macronutrient or micronutrient and its value may depend on, e.g., a relevance given by the nutritionist to the specific macronutrient or micronutrient for the considered user.

By applying equation [3] to the partial score vector PSV and the weights vector WGT reported above, the value of the partial compliance score Sₙ of the considered food corresponds to 87.12.

With reference again to equation [1], in the following lines, the computation of the weight Wᵣ will be described.

According to the present invention, for each rule in the user diet pattern, the processing module 10 calculates a value Wᵣ(j), where j=1, . . . , M and M is the number of rules in the user diet pattern. Each value Wᵣ(j) indicates the compliance of the considered food intake with the relevant j-th rule. The value Wᵣ to be applied in equation [1] is then computed by the processing module 10 by performing a weighted average of all values Wᵣ(j), j=1, . . . , M, according to the following equation:

$${W}_{r} = \frac{{\sum }_{j = 1}^{N} K ( j ) \times {W}_{r} ( j )}{{\sum }_{j = 1}^{N} K ( j )} \qquad [ 4 ]$$

where each K(j), j=1, . . . , M is a constant, preferably comprised in the range [0, 1], which depends on the food category in a respective food rule and can be determined using the following Table 9. In Table 9, the first column reports the food category and the second column the corresponding value of the constant K(j).

TABLE 9

| Food Category | K values |
|---|---|
| Milk | 0.03 |
| Biscuits | 0.01 |
| Honey | 0.03 |
| Fresh Fruit | 0.0001 |
| Yogurt | 0.03 |
| Pasta | 0.001 |
| Vegetables | 0.0001 |
| Meat | 0.003 |
| Salad | 0.0001 |
| Oil | 0.03 |
| Bread | 0.001 |
| Wine | 0.1 |
| Rice | 0.0001 |
| Cereals | 0.001 |
| Potatoes | 0.0001 |
| Fish | 0.00003 |
| Eggs | 0.001 |
| Cold Cuts | 0.1 |
| Fresh Legumes | 0.0003 |
| Dried Legumes | 0.001 |
| Soy Milk | 0.0001 |
| Seasoned Cheeses | 0.001 |
| Fresh Cheeses | 0.01 |
| Butter | 0.01 |
| Beer | 0.03 |
| Cakes | 0.1 |
| Jam | 0.01 |
| Tofu or other soy products | 0.0001 |
| Sugar | 0.01 |
| Other meats | 0.001 |

For sake of simplicity, it is considered a rule in the diet pattern that specifies that the user may take 2 or 3 portions per day of food in the category “Bread” and that the reference intake is 60 g. The following description will focus on computing the weight Wᵣ(j) corresponding to this exemplary rule. This is not limiting as the computation of the weights for other rules is analogous.

According to the present invention, the weight Wᵣ(j) for the rule above is computed by the processing module 10 at a given date and time of the day corresponding to the user food intake (the given date and time of the day will be referred to as “evaluation time”). For sake of example, it is assumed that the evaluation time is 2016-02-08, 12:30 (see the exemplary food diary table of Table 4 herein above). The processing module 10 preferably retrieves the food diary table from the user database 113 and, on the basis of the category of food specified in the rule (i.e. “Bread” for the example above) and on the frequency of intake specified in the rule (i.e. “daily”), it preferably extracts the user food intake before the evaluation time and the user food intake at the evaluation time. Then, the processing module 10 preferably computes the following values:
a history intake quantity HIQ indicating the food consumed by the user for the considered category of food (“Bread”) before the evaluation time;
a food intake quantity FIQ indicating the food consumed by the user for the considered category of food (“Bread”) at the evaluation time;
a minimum ideal quantity MINQ indicating the minimum ideal quantity of food to be consumed in accordance with the considered rule. It is preferably computed as the product of the minimum number of portions specified in the considered rule and the reference intake. Moreover, as it will be described herein after, it is preferably updated on the basis of the food consumed by the user before the evaluation time;
a maximum ideal quantity MAXQ indicating the maximum ideal quantity of food to be consumed according to the considered rule. It is preferably computed as the product of the maximum number of portions specified in the considered rule and the reference intake. Moreover, as it will be described herein after, it is preferably updated on the basis of the food consumed by the user before the evaluation time.
   In particular, the history intake quantity HIQ is computed according to the following equation:

$$HIQ = {\sum }_{k = 0}^{R} NP ( k ) \times Q ( k ) \qquad [ 5 ]$$

where R is the number of rows of the food diary table relating to intakes of food in the considered food category before the evaluation time, NP(k) is the value of the portion field in the food diary table in the k-th row, k=1, . . . , R, and Q(k) is the corresponding quantity of the portion.

The food intake quantity FIQ is computed according to the following equation:

FIQ=NP×Q [6]

where NP is the number of portions of food in the considered food category consumed by the user at the evaluation time and Q is the corresponding quantity of food per portion.

For example, considering the food diary table of Table 4 above, the HIQ value for the “Bread” food category corresponds to 60 g. Moreover, considering the exemplary rule mentioned above, the minimum ideal quantity MINQ is equal to 2×60 g=120 g and the maximum ideal quantity MAXQ is equal to 3×60 g=180 g.

According to the present invention, the minimum ideal quantity MINQ and the maximum ideal quantity MAXQ are updated on the basis of the food consumed by the user before the evaluation time. In particular, the processing module 10 preferably updates the minimum ideal quantity MINQ according to the following equations:

$$MINQ = \{ \begin{matrix}MINQ - HIQ , & if MINQ \geq HIQ \\ 0 , & otherwise\end{matrix} \qquad [ 7 ]$$

and it preferably updates the maximum ideal quantity MAXQ according to the following equations:

$$MAXQ = \{ \begin{matrix}MAXQ - HIQ , & if MAXQ \geq HIQ \\ 0 , & otherwise\end{matrix} . \qquad [ 8 ]$$

According to the example above, the minimum ideal quantity MINQ at the evaluation time for the “Bread” food category is 60 g, the maximum ideal quantity MAXQ at the evaluation time is 120 g and the food intake quantity FIQ is 60 g.

The weight Wᵣ(j) is then computed according to the following equations:

$${W}_{r} ( j ) = \{ \begin{matrix}F ( t ) , & if FIQ \geq MINQ \\ 1 , & if FIQ ∈ [ MINQ , MAXQ ] \\ {e}^{k ( MAXQ - FIQ )} , & if FIQ > MAXQ\end{matrix} \qquad [ 9 ]$$

where k is a numerical constant preferably comprised in the range [0, 1] whose value can be determined using Table 9, and F(t) is a function of time defined as follows:

$$F ( t ) = \{ \begin{matrix}1 , & if MINQ = 0 \\ 1 - h \cdot {t}^{10 / min} , & if MINQ > 0\end{matrix} \\ where \text{:} \\ h = \frac{1}{{duration}^{10 / min}} \\ and \\ min = \frac{MINQ}{2 \cdot referenceIntake} \qquad [ 10 ]$$

where “duration” is a parameter corresponding to the frequency of intake in the rule (e.g. daily, weekly) and “referenceIntake” is the reference quantity of intake specified in the rule.

According to the example above, Wr(j) at the evaluation time corresponds to value 1 as FIQ belongs to the range defined by the updated values of the minimum ideal quantity MINQ and the maximum ideal quantity MAXQ.

Once the partial compliance score Sₙ and the weight Wᵣ are computed by applying equations [3] and [4] above, respectively, the compliance score of the considered food is computed by applying equation [1] above.

Referring back to the flowchart of FIG. 3, once the compliance score is computed (step 306), the processing module 10 preferably sends it to the user's device 131 (step 307).

In this way, advantageously, the system of the present invention provides the user with a clear indication about the compliance of the consumed food (or, before having a meal, of a food that the user intends to consume) with her/his diet pattern.

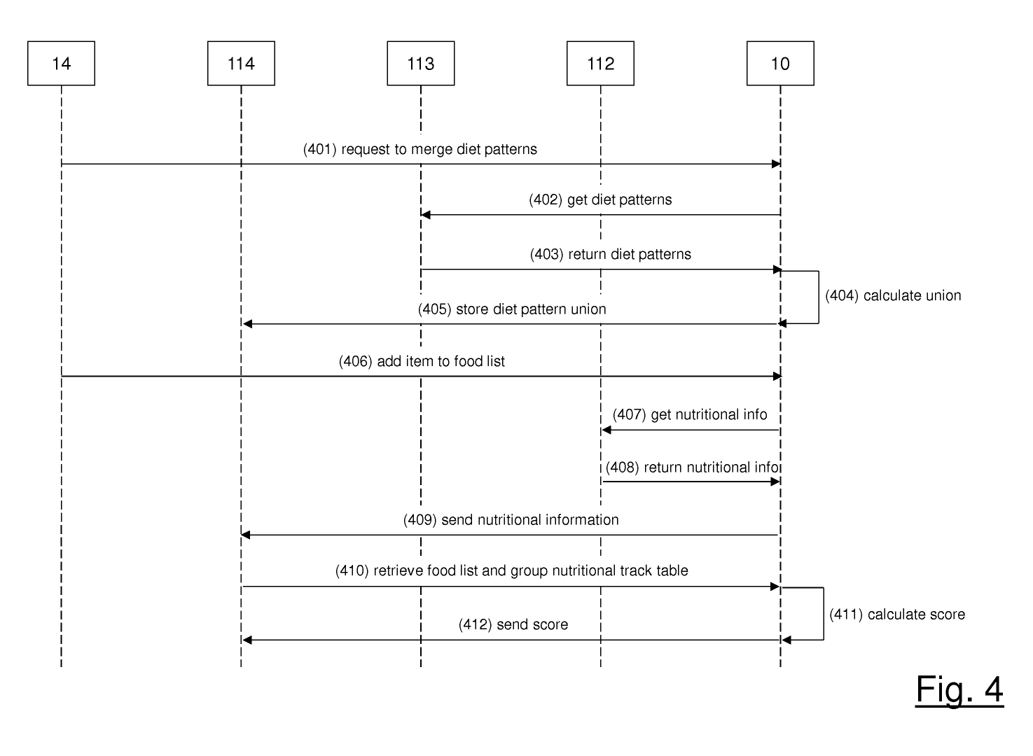

FIG. 4 is a flow chart illustrating a third use case of application of the present invention. According to this third use case, a group of users 14 (for instance, a sport team) share their diet patterns that are merged by the processing module 10 in a diet pattern union using the diet pattern union algorithm, which will be described herein after. The diet pattern union may be used, for instance, for purchasing foods at a supermarket for the whole group.

Indeed, according to the present invention, the processing module 10 may calculate a compliance score indicating the compliance of a list of foods with the calculated diet pattern union. Advantageously, this allows assuring that the nutritional needs of each user of the group 14 are taken into consideration within the list of foods and that only an appropriate quantity of food is actually purchased, so that waste is avoided.

More in detail, in FIG. 4, at step 401, the users of the group 14 send a request to the processing module 10 to merge their diet patterns. At step 402, the processing module 10 accesses the user database 113 to get the individual diet patterns of the users and, at step 403, the user database 113 returns them to the processing module 10. At step 404, the processing module 10 preferably computes the diet pattern union for the group 14.

According to the present invention, the diet pattern union of the group 14 preferably comprises nutritional information that are derived from the nutritional information of all the diet patterns of the users of the group: in particular, the values of the reference intake, maximum quantity of intake and minimum quantity of intake of each macronutrient in the individual diet patterns and the values of the reference intake, maximum quantity of intake and minimum quantity of intake of each micronutrient in the individual diet patterns are summed. In other words, the diet pattern union preferably comprises the union of macronutrients and micronutrients of the individual diet patterns of the users of the group, and the macronutrients and micronutrients in the diet pattern union are associated with values that are the sum of the corresponding quantities in the individual diet patterns.

Moreover, the diet pattern union preferably comprises a number of bioactive compounds that results from the union of the bioactive compounds comprised in the individual diet patterns of the users of the group. Also in this case, the maximum quantity of intake and minimum quantity of intake of each bioactive compound in the diet pattern union is computed as the sum of the corresponding quantities in the individual diet patterns.

As far as intolerances and/or allergies are considered, the diet pattern union preferably comprises only foods that are mentioned in all the individual diet patterns of the users of the group (i.e. the diet pattern union preferably comprises, as far as intolerances and/or allergies are considered, only foods to which all the users of the group are intolerant and/or allergic).

The union of the food rules is preferably performed as follows. If a first food rule is comprised within a first diet pattern of a user of the group and a second food rule is comprised within a second diet pattern of another user of the group, the first food rule comprising, for a given food category, a first reference quantity of intake and the second rule comprising, for the same food category, the same reference quantity of intake, a rule is added to the diet pattern union containing, for the given food category, the reference quantity of intake of the first rule and the second rule. The minimum number of portions and the maximum number of portions of this food rule in the diet pattern union are calculated by transforming, if necessary, the frequencies of intake of the first rule and the second rule in a common frequency of intake (e.g. weekly), determining, for each rule, a minimum number of portions and a maximum number of portions according to the common frequency of intake, and summing those numbers.

In the following lines an example of obtaining a rule for the union diet pattern will be described. A first rule for a given food category of a first diet pattern specifies that the frequency of intake is “daily”, that the minimum number of portions is 1 and the maximum number of portions is 2. A second rule for the same food category of a second diet pattern specifies that the frequency of intake is “weekly” and that the minimum number of portions as well as the maximum number of portions is 3. The union of the first rule and the second rule is computed as follows:
a common frequency of intake is established, namely “weekly”;
according to the first rule, the weekly minimum number of portions is 7 and the weekly maximum number of portions is 14;
the weekly minimum number of portions and the weekly maximum number of portions according to the first rule and the second rule are summed, so that the corresponding rule in the diet pattern union specifies that the frequency of intake is “weekly”, the minimum number of portions is 10 and the maximum number of portions is 17.
   All the other food rules comprised in the individual diet patterns are added as such to the diet pattern union.

As an example, Table 10 herein below reports a diet pattern union that can be obtained according to the present invention merging the diet pattern for the “Mediterranean diet” and the diet pattern for the “Dash diet” reported in Table 1 and Table 2, respectively.

TABLE 10
Union of Mediterranean and Dash diet patterns

| Nutritional composition: | | | | Ref | Max | Min | Unit |
|---|---|---|---|---|---|---|---|
| Energy | | | | 4000 | | | Kcal |
| | Protein | | | | 185.36 | 130.98 | g |
| | Carbohydrates | | | | 556.83 | 485.49 | g |
| | Fat | Sat | | | 40.03 | 30.70 | g |
| | Fat | Unsat | Mono | | 80.94 | 64.03 | g |
| | Fat | | Poly | | 32.92 | 25.43 | g |

| Dietary Total Fibres | | | 98.75 | 67.97 | g |
|---|---|---|---|---|---|
| Vitamins | Soluble | Thiamin | 5.66 | 3.30 | mg |
| | | Riboflavin | 4.42 | 3.52 | mg |
| | | Niacin | 54.53 | 35.40 | mg |
| | | Pantothenic acid | 6.47 | 4.01 | mg |
| | | Vitamin B6 | 7.42 | 5.26 | mg |
| | | Biotin | 56.21 | 37.59 | μg |
| | | Folic acid | 960.68 | 690.81 | μg |
| | | Vitamin B12 | 9.34 | 4.06 | μg |
| | | Vitamin C | 417.61 | 276.29 | mg |
| | Fatsoluble | Vitamin A | 3020.33 | 2087.77 | μg |
| | | Vitamin D | 10.09 | 5.91 | μg |
| | | Vitamin E | 52.84 | 42.08 | mg |

| Water | | 5480.49 | 4681.75 | g |
|---|---|---|---|---|
| Sugars | | 232.84 | 198.13 | g |
| Alcohol | | 10.00 | 0.00 | g |
| Minerals | Calcium | 2382.60 | 1850.49 | mg |
| | Chlorine | 2748.25 | 1561.25 | mg |
| | Iron | 37.97 | 30.33 | mg |
| | Iodine | 373.58 | 239.21 | μg |
| | Magnesium | 307.77 | 364.41 | mg |
| | Phosphorus | 4637.20 | 3191.30 | mg |
| | Potassium | 9536.73 | 7320.57 | mg |
| | Selenium | 120.49 | 44.60 | μg |
| | Manganese | 3.76 | 2.93 | mg |
| | Zinc | 36.09 | 23.75 | mg |
| | Sodium | 4971.18 | 3073.91 | mg |
| | Copper | 2.62 | 1.70 | mg |

| Bioactive compounds: | Min | Max | Unit |
|---|---|---|---|
| Omega3 | 4 | 8 | g |
| Ascorbic acid | 120 | 180 | mg |
| Carotenoid | 12 | 36 | mg |
| Isoflavones | 120 | 160 | mg |
| Polyphenols | 2 | 6 | g |
| Quercetin | 100 | 600 | mg |
| Resveratrol | 400 | 800 | mg |
| Lycopene | 2 | 30 | mg |
| Folic acid | 0.4 | 0.8 | mg |
| Lutein | 12 | 40 | mg |
| Fiber | 50 | 60 | g |

| Food rules: | | | | | |
|---|---|---|---|---|---|
| Min | Max | Freq | Food | Ref | Unit |
| 2 | 2 | daily | Vegetables | 250 | g |
| | | | Salad | 50 | g |
| 3 | 3 | daily | Fresh fruit | 150 | g |
| | | | Dry fruit | 30 | g |
| 2 | 3 | daily | Bread | 50 | g |
| 1 | 1 | daily | Pasta | 80 | g |
| | | | Rice | 80 | g |
| 1 | 1 | daily | Biscuits | 20 | g |
| 1 | 2 | weekly | Potatoes | 200 | g |
| 3 | 5 | weekly | Meat | 100 | g |
| 2 | 3 | weekly | Fish | 150 | g |
| 1 | 2 | weekly | Eggs | 60 | g |
| 0 | 3 | weekly | Cold cuts | 50 | g |
| 2 | 2 | weekly | Fresh | 100 | g |
| | | | legumes | | |
| | | | Dry | 30 | g |
| | | | legumes | | |
| 14 | 14 | weekly | Milk | 125 | g |
| | | | Yogurt | 125 | g |
| 0 | 4 | weekly | Seasoned | 50 | g |
| | | | cheese | | |
| | | | Fresh | 100 | g |
| | | | cheese | | |
| 0 | 5 | weekly | Butter | 10 | g |
| 2 | 3 | daily | Oil | 10 | g |
| 0 | 1 | daily | Wine | 100 | g |
| | | | Beer | 100 | g |
| 0 | 3 | daily | Sugar | 5 | g |
| | | | Honey | 5 | g |
| 0 | 1 | weekly | Cakes | 100 | g |
| 0 | 3 | weekly | Jam | 7 | g |
| 0 | 3 | daily | Soy milk | 125 | g |
| 7 | 8 | daily | Cereals | 30 | g |
| | | | Bread | 30 | g |
| | | | Pasta | 65 | g |
| | | | Rice | 65 | g |
| 4 | 5 | daily | Vegetables | 250 | g |
| 4 | 5 | daily | Fresh fruit | 150 | g |
| 0 | 6 | daily | Meat | 30 | g |
| | | | Fish | 30 | g |
| | | | Eggs | 60 | g |
| 4 | 5 | weekly | Dry fruits | 30 | g |
| | | | Fresh | 65 | g |
| | | | legumes | | |
| 2 | 3 | daily | Oil | 15 | g |
| 0 | 5 | weekly | Sugar | 15 | g |
| | | | Jam | 15 | g |

After having computed the diet pattern union as described above (step 404), at step 405 the processing module 10 preferably associates the diet pattern union with the considered group 14 and stores the diet pattern union into the group database 114.

At this point, the group 14 may require the system 1 to compute a compliance score of a list of foods with its diet pattern union. Specifically, when an item is added to the food list by a user, it is communicated to the processing module 10 through a user's device 141 (step 406), and the processing module 10 preferably accesses the food database 112 to get detailed nutritional information about the food and to get the food category (step 407). At step 408, the food database 112 returns the nutritional information and the food category to the processing module 10. Then, at step 409, the processing module 10 sends the nutritional information and the food category information to the group database 114 to insert the added item in the food list table and to update the group nutrition track table. At step 410, the processing module 10 preferably retrieves the updated food list table and group nutritional track table and, on the basis of the data contained therein, calculates the compliance score of the food list with the diet pattern union of the group (step 411). This computation is performed by applying the food list compliance algorithm that will be described in detail herein after.

According to the present invention, the food list compliance algorithm computes a compliance score of a list of foods with a diet pattern, e.g. the diet pattern union of a group 14 of users according to the use case considered herein above. Similarly, the same algorithm may be used to compute a compliance score of a list of foods with a user diet pattern.

The algorithm preferably calculates a compliance score as an integer number within a given compliance range. The compliance score may be for instance comprised between 0 and 100. The minimum value of the compliance score, e.g. 0, is returned by the algorithm for a food list that is not compliant with the diet pattern. The maximum value of the compliance score, e.g. 100, is returned by the algorithm for a food list that is completely compliant with the diet pattern, the term “completely” meaning that the food list is compliant with the diet pattern with respect to both the nutritional composition and the food rules. The food list compliance algorithm preferably also computes a controller vector C indicating in percentage, for each food category, if the foods in the food list exceed a “right” quantity or if they are insufficient, as it will be described in greater detail herein after.

For computing the compliance score, the processing module 10 preferably calculates, on the basis of the diet pattern, two vectors: an ideal mean value nutritional vector INV(TP)μ and an ideal variance nutritional vector INV(TP)σ. The ideal mean value nutritional vector INV(TP)μ preferably comprises ideal average intakes of each macronutrient and micronutrient specified in the diet pattern, computed for a time period TP. The time period TP indicates the number of days for which the foods in the food list shall be sufficient, and it may be provided to the processing module 10 by the group 14 when requesting to compute the compliance score. The ideal average intake of a macronutrient or a micronutrient is preferably computed using the maximum quantity of intake and minimum quantity of intake, reported in the diet pattern (such as, for instance, the diet pattern of Table1, Table 2 or Table 10). The ideal variance nutritional vector INV(TP)σ preferably comprises the standard deviations from the ideal average intakes specified in the ideal mean value nutritional vector INV(TP)μ. The standard deviation for each macronutrient or micronutrient is preferably computed according to the values of minimum intake and maximum intake indicated in the diet pattern.

As an example, referring to the diet pattern union exemplified above in Table 10, an ideal mean value nutritional vector INV(TP)μ for a time period of 7 days, may be represented as follows:
INV(7-days)μ=[energy_{μ}, protein_{μ}, carbohydrate_{μ}, saturatedFattyAcids_{μ}, monounsaturated_{μ}, polyunsaturated_{μ}, dietaryTotalFibre_{μ}, thiamin_{μ}, riboflavin_{μ}, niacin_{μ}, pantothenicAcid_{μ}, vitamin B6_{μ}, biotin_{μ}, folicAcid_{μ}, vitamin B12_{μ}, vitaminC_{μ}, vitaminA_{μ}, vitaminD_{μ}, vitamin E_{μ}, water_{μ}, sugars_{μ}, alcohol_{μ}, calcium_{μ}, chlorine_{μ}, iron_{μ}, iodine_{μ}, magnesium_{μ}, phosphorus_{μ}, potassium_{μ}, selenium_{μ}, manganese_{μ}, zinc_{μ}, sodium_{μ}, copper_{μ}, ascorbicAcid_{μ}, carotenoid_{μ}].
   Inserting the corresponding numerical values, the vector is:
INV(7-days)μ=[28000, 1122.555, 3648.12, 247.555, 507.395, 204.225, 583.52, 31.36, 27.79, 314.755, 36.68, 44.38, 328.3, 5780.215, 46.9, 2428.65, 17878.35, 56, 332.22, 35567.84, 1508.395, 35, 14815.815, 15083.25, 240.45, 2144.765, 3054.205, 27399.75, 59000.9, 577.815, 10257.66, 209.44, 28157.815, 15.12, 1050, 168]
   Moreover, for the same example of diet pattern of Table 10, an ideal variance nutritional vector INV(TP)σ for a time period of 7 days, may be represented as follows:
INV(7-days)σ=[energy_{σ}, protein_{σ}, carbohydrate_{σ}, saturatedFattyAcids_{σ}, monounsaturated_{σ}, polyunsaturated_{σ}, dietaryTotalFibre_{σ}, thiamin_{σ}, riboflavin_{σ}, niacin_{σ}, pantothenicAcid_{σ}, vitamin B6_{σ}, biotin_{σ}, folicAcid_{σ}, vitamin B12_{σ}, vitaminC_{σ}, vitaminA_{σ}, vitaminD_{σ}, vitaminE_{σ}, water_{σ}, sugars_{σ}, alcohol_{σ}, calcium_{σ}, chlorine_{σ}, iron_{σ}, iodine_{σ}, magnesium_{σ}, phosphorus_{σ}, potassium_{σ}, selenium_{σ}, manganese_{σ}, zinc_{σ}, sodium_{σ}, copper_{σ}, ascorbicAcid_{σ}, carotenoid_{σ}].
   Inserting the corresponding numerical values, the vector is:
INV(7-days)σ=[1400, 191.905, 249.69, 32.655, 59.185, 26.215, 107.73, 8.26, 3.15, 66.955, 8.61, 7.56, 65.17, 944.545, 18.48, 494.62, 3263.96, 14.63, 37.66, 2795.59, 121.485, 35, 1862.385, 4154.5, 25.34, 470.295, 500.185, 5060.65, 7756.21, 265.615, −10231.34, 43.19, 6640.445, 3.22, 210, 84].
   Then, for each macronutrient and micronutrient of the diet pattern, the processing module 10 preferably determines a Gaussian function (with module equal to 1) analogously to what has been described above with reference to the compliance score algorithm. The Gaussian function is the function of equation [2], wherein for each macronutrient and micronutrient of the diet pattern, the mean value μ is the ideal average intake specified in the ideal mean value nutritional vector INV(TP)μ and the variance σ is the ideal variance specified in the ideal variance nutritional vector INV(TP)σ. The Gaussian function is used to calculate a partial compliance score vector PSV for the considered food list, as it will be described herein below. For example, it is assumed to consider the following food list:

| Category | Food | Quantity [g] |
|---|---|---|
| Vegetables | Spinach | 3570 |
| Vegetables | Green Salad | 3080 |
| Fresh Fruit | Apples | 8400 |
| Bread | Bread | 2730 |
| Rice | Rice | 560 |
| Biscuits | Biscuits | 140 |
| Potatoes | Potatoes | 400 |
| Meat | Meat | 1550 |
| Cold Cuts | Ham | 100 |
| Fresh Legumes | Chickpeas | 460 |
| Milk | Milk | 1750 |
| Seasoned Cheeses | Parmesan | 100 |
| Butter | Butter | 20 |
| Oil | Oil | 525 |
| Wine | Wine | 700 |
| Sugar | Sugar | 115 |
| Cakes | Apple pie | 100 |
| Jam | Apple jam | 14 |

The processing module 10 preferably retrieves from the group database 114 the group nutrition track table and determines a nutritional vector NV that comprises the intakes of foods contained in the food list. The nutritional vector NV preferably comprises the contributions in term of macronutrients and micronutrients as stored in the group database 114 (in particular, in the group nutrition track table) for the food contained in the food list. The nutritional vector NV may be represented as follows:
NV=[energy, protein, carbohydrate, saturatedFattyAcids, monounsaturated, polyunsaturated, dietaryTotalFibre, thiamin, riboflavin, niacin, pantothenicAcid, vitamin B6, biotin, folicAcid, vitamin B12, vitaminC, vitaminA, vitaminD, vitaminE, water, sugars, alcohol, calcium, chlorine, iron, iodine, magnesium, phosphorus, potassium, selenium, manganese, zinc, sodium, copper, ascorbicAcid, carotenoid].
   In the example the nutritional vector NV is as follows:
NV=[27597.98 998.68 3950.923 238.943 510.9575 133.891 516.798 12.1038 35.4572 167.295 6.413 26.258 205.05 7079.8 6.65 3241.12 18594.75 24.935 270.045 33387.89 1220.053 74.9 11660.17 2664.4 292.751 542.1 4336.4 13823.68 63295.6 410.5 219.44 220.436 53660.13 18.5925 1862.12 151.41616]
   The processing module 10 preferably applies the Gaussian functions defined above to the respective components of the nutritional vector NV and calculates the partial compliance score vector PSV. Referring to the example above, the partial compliance score vector is:
PSV=[99.54, 97.71, 92.15, 99.61, 99.98, 67.04, 97.89, 73.94, 71.95, 76.38, 50.33, 72.67, 81.98, 90.02, 76.83, 86.08, 99.73, 77.84, 85.95, 96.68, 73.13, 93.03, 85.26, 60.87, 78.93, 52.46, 69.41, 67.04, 98.31, 97.82, 94.79, 99.64, 44.07, 93.74, 43.57, 99.78],
   wherein each component of the partial compliance score vector PSV is the partial compliance score for the corresponding macronutrient or micronutrient of the considered diet pattern.

The compliance score of the list of foods with the diet pattern is then calculated by performing a weighted average of the partial compliance scores contained in the partial compliance score vector PSV. The weighted average is preferably computed by multiplying each component of the partial compliance score vector PSV by a respective weight of a weight vector WGT′. The weight vector may be for example the following one:
WGT′=[2, 3, 2, 2, 2, 2, 2, 2, 2, 2, 2, 2, 2, 2, 2, 2, 2, 2, 2, 2, 2, 2, 4, 2, 4, 2, 2, 2, 5, 2, 2, 2, 5, 2, 2, 2].

Considering for instance the partial compliance score vector PSV and the weights reported herein above, the compliance score for the considered food list is equal to 81.27.

The controller vector C is preferably determined by the processing module 10 according to the following procedure:
for each food category, it calculates a minimum right quantity MIN_RQ and a maximum right quantity MAX_RQ according to the time period TP. The minimum right quantity MIN_RQ and maximum right quantity MAX_RQ for a food category are computed by using the food rules defined in the considered diet pattern. For instance, the minimum right quantity MIN_RQ may be determined for a given food category by multiplying the minimum number of portions of food in that category by the reference intake and by the time period TP, while the maximum right quantity MAX_RQ may be determined for a given food category by multiplying the maximum number of portions of food in that category by the reference intake and by the time period TP. For instance, if for the category “Vegetables”, the minimum number of portions is 2, the maximum number of portions is 3, the frequency of intake is daily, the reference intake is 130 g, and the time period TP is 7 days, the minimum right quantity MIN_RQ is equal to 2×130 g×7=1820 g, while the maximum right quantity MAX_RQ is equal to 3×130 g×7=2730 g.
For each food category, the processing module 10 preferably computes the respective component of the controller vector C by applying the following equations:

$$f ( x ) = \{ \begin{matrix}100 , & if x ∈ [ MIN\_RQ , MAX\_RQ ] \\ \frac{x}{MIN\_RQ} \cdot 100 - 100 , & if x < MIN\_RQ \\ \frac{x}{MAX\_RQ} \cdot 100 - 100 , & if x > MAX\_RQ\end{matrix} \qquad [ 11 ]$$

where x is the quantity of foods of the given food category contained in the food list. If, for instance, the food list contains 1500 g of vegetables, considering the exemplary values of the minimum right quantity MIN_RQ and maximum right quantity MAX_RQ computed above, the component related to the “Vegetables” food category in the controller vector C is equal to −17.6%.
   The system of the present invention may be used in different situations. For instance, the system may advantageously support a user in the selection of a food at the supermarket or at the restaurant, allowing her/him to easily verify whether the food and the relevant intake are appropriate to her/his nutritional needs, i.e. whether they are compliant with her/his diet pattern. The procedure is automatic and easily accessible by the user by means, for example, of a mobile application installed on her/his mobile device.

The system allows to provide in a flexible manner a dietary model to the user (the diet pattern) that is not a diet in a strict sense as it does not provide any constraint about foods. The system according to the present invention also provides the user with a method that allows the user to select a food and immediately verify whether the food is compliant with her/his diet pattern or not. The user is hence left with a certain degree of freedom in the selection of foods as she/he is not bound to strict rules and prohibitions. This may help the user to adopt healthy dietary habits while avoiding the risk that the user gets bored with restrictions. Moreover, the system of the present invention advantageously helps users to easily verify whether the foods that they have at home or they may find at a restaurant or at a canteen are compliant with their diet pattern.

Furthermore, the system of the present invention allows to determine diet patterns suitable for groups of users, such as families or sport teams, in an automatic way and to check the compliance of a shopping list of foods with a user's diet pattern or with the group's diet pattern.

Finally, it should be noticed that the system of the present invention may be used for supporting a user in the selection of food not only in the situation wherein the user is assumed to be the consumer of the food, but also in a situation wherein the user is selecting food for another person or even for an animal (e.g. a domestic animal, a racer). For instance, for getting a customized diet pattern, the user provides to the nutritionist profile data relating to the other person or animal and the customized diet pattern described above meets the profile of the other person or animal, namely it comprises the nutritional information and the food rules necessary to satisfy the corresponding nutritional needs.

1. A system for supporting a user in the selection of food, said system comprising a processing module configured to:
determine a diet pattern of said user, said diet pattern comprising nutritional information related to a number of macronutrients or micronutrients or bioactive compounds into which food may be decomposed and corresponding values of daily intakes, said diet pattern further comprising a number of food rules, each food rule comprising at least one food category and, for each food category, a respective frequency of intake and a respective reference quantity of intake; and
receive from said user information indicating a target food, compute a compliance score of said target food with said diet pattern and notifying said user of said compliance score.

2. The system according to claim 1, further comprising a diet pattern database comprising a set of pre-determined diet patterns, wherein said processing module is further configured to
receive a user's profile comprising user-related information, the user-related information comprising one or more of: gender, height, weight, body mass index, age, diseases, intolerances, allergies;
select one diet pattern of said set of pre-determined diet patterns; and
customize said selected diet pattern on the basis of said user's profile.

3. The system according to claim 1, wherein it further comprises a food database comprising a number of entries relating to foods and, for each entry, the respective food category and nutritional information comprising quantities of said number of macronutrients or micronutrients or bioactive compounds for a reference amount of the respective food.

4. The system according to claim 1, wherein it further comprises a user database configured to store:
a food diary of the user comprising information about the foods consumed by said user subdivided in a number of food categories; and
a user nutrition track table comprising nutritional information of each food consumed by said user, said nutritional information comprising said macronutrients or micronutrients or bioactive compounds.

5. The system (1) according to claim 3, wherein the processing module is further configured to, upon reception from said user of said information indicating a target food, wherein said information indicating a target food comprises a target food intake:
access said food database to get the food category of said target food and the nutritional information related to the reference amount of said target food;
compute the nutritional information related to said target food intake; and
access said user database to update said food diary and said user nutrition track table with said information indicating the target food and said computed nutritional information.

6. The system according to claim 1, wherein said processing module is further configured to compute said compliance score as product of a partial compliance score indicating the compliance of said target food with respect to the nutritional information of said user diet pattern and a weight value indicating whether said target food fulfils the food rules of said user diet pattern.

7. The system according to claim 5, wherein said processing module is further configured to compute said partial compliance score by:
computing an ideal nutritional vector comprising ideal average daily intakes of said macronutrients or micronutrients, each of said ideal average daily intakes being computed as an average value between a minimum quantity of intake and a maximum quantity of intake comprised in said values of daily intakes of said user diet pattern,
computing an ideal variance nutritional vector comprising standard deviations from said ideal average daily intakes;
computing a further ideal nutritional vector on the basis of the time of the day at which said computation is performed, said further ideal nutritional vector comprising percentage values of said ideal average daily intakes indicating the percentages of said ideal average daily intakes that could have been consumed at said time of the day by said user;
computing a nutritional intake vector on the basis of the user nutrition track table, said nutritional intake vector comprising intakes of said macronutrients or micronutrients at said time of the day;
for each component of said nutritional intake vector, creating a Gaussian function whose mean value is the corresponding component of said further ideal nutritional vector and whose standard deviation is the corresponding component of said ideal variance nutritional vector, applying each Gaussian function to the respective component of said nutritional intake vector and obtaining a partial score vector comprising individual compliance scores of said macronutrients or micronutrients; and
computing said partial compliance score as a weighted sum of said individual compliance scores.

8-15. (canceled)

16. The system according to claim 6, wherein said processing module is further configured to compute said weight value as a weighted sum of individual weight values, each individual weight value indicating the compliance of said target food intake with a respective food rule of said number of food rules in the user diet pattern.

17. The system according to claim 16, wherein said processing module is further configured to compute said individual weight value Wᵣ(j) as:
$${W}_{r} ( j ) = \{ \begin{matrix}F ( t ) , & if FIQ \geq MINQ \\ 1 , & if FIQ ∈ [ MINQ , MAXQ ] \\ {e}^{k ( MAXQ - FIQ )} , & if FIQ > MAXQ\end{matrix}$$
where FIQ is a food intake quantity indicating the food consumed by the user for the food category of said target food at said time of the day, MINQ is a minimum ideal quantity of food to be consumed in accordance with said respective food rule; MAXQ is a maximum ideal quantity of food to be consumed in accordance with said respective food rule, k is a numerical constant comprised in the range [0, 1] and F(t) is a function of time defined as:
$$F ( t ) = \{ \begin{matrix}1 , & if MINQ = 0 \\ 1 - h \cdot {t}^{10 / min} , & if MINQ > 0\end{matrix} \\ where \text{:} \\ h = \frac{1}{{duration}^{10 / min}} \\ and \\ min = \frac{MINQ}{2 \cdot referenceIntake}$$
where duration is a parameter corresponding to the frequency of intake comprised in the respective food rule and referenceIntake is the reference quantity of intake comprised in the respective food rule.

18. The system according to claim 1, wherein said processing module is further configured to merge the diet patterns of at least two users into a diet pattern union.

19. The system according to claim 18, wherein said processing module is further configured to compute said diet pattern union as the union of said macronutrients or micronutrients or bioactive compounds of the diet patterns of the at least two users, wherein the nutritional information of said macronutrients or micronutrients or bioactive compounds in the diet pattern union is associated with the sum of the corresponding nutritional information in the diet patterns of said at least two users.

20. The system according to claim 19, wherein said processing module is further configured to compute a food rule in said diet pattern union as the union of corresponding food rules in said diet patterns of said at least two users, wherein said corresponding food rules comprise a same food category, a same reference quantity of intake and respective minimum numbers of portions and maximum numbers of portions, by:
transforming the frequencies of intake of said corresponding food rules in a common frequency of intake;
adjusting said minimum number and said maximum number of portions, for each of said corresponding food rules, according to said common frequency;
summing said adjusted minimum numbers of portions; and
summing said adjusted maximum numbers of portions.

21. The system according to claim 1, wherein the processing module is further configured to receive from said user a list of foods and to compute a further compliance score of said list of foods with said diet pattern.

22. A method for supporting a user in the selection of food, said method comprising:
a) determining a diet pattern for said user, said diet pattern comprising nutritional information of a number of macronutrients or micronutrients or bioactive compounds into which food may be decomposed, said diet pattern further comprising a number of food rules, each food rule comprising at least one food category and, for each food category, a frequency of intake and a corresponding quantity of intake; and
b) computing a compliance score of a food indicated by the user with said diet pattern; and
c) notifying said user of said compliance score.

23. A non-transitory computer-readable medium encoded with computer-readable instructions that, when executed by a computer, cause the computer to perform the method according to claim 22.
